(19) United States
(12) Patent Application Publication
(10) Pub. No.: US 2006/0134008 A1
(43) Pub. Date: Jun. 22, 2006

(54) Compositions and methods for pulmonary conditions

(76) Inventor: Daniel Deaver, Frankiln, MA (US)

Correspondence Address: ELMORE PATENT LAW GROUP, PC, 209 MAIN STREET, N. CHELMSFORD, MA, 01863, US

(21) Appl. No.: 11/302,526

(22) Filed: Dec. 13, 2005

Related U.S. Application Data
(60) Provisional application No. 60/636,755, filed on Dec. 16, 2004.

(51) Int. Cl. A61K 31/4745; A61K 9/14; A61K 31/24; A61L 9/04

(52) U.S. Cl. 424/46; 514/291; 514/536

(57) ABSTRACT

Compositions and methods for the treatment of pulmonary conditions, especially pulmonary conditions characterized by persistent cough, are disclosed. The compositions and methods employ at least one muscarinic receptor antagonists and at least one local anesthetic administered pulmonarily either simultaneously or in sequence. The compositions may be in powder or liquid form.

### RELATED APPLICATION

This application claims the benefit of U.S. Provisional Application No. 60/636,755, filed on Dec. 16, 2004. The entire teaching of the above application is incorporated herein by reference.

### BACKGROUND OF THE INVENTION

The successful treatment of pulmonary conditions such as persistent cough, asthma and chronic obstructive pulmonary disorder (COPD) present continuing difficulties. At least one line of research has suggested the administration of a topical anesthetic to quell the discomfort and symptoms. For example, Gleich et al. propose methods to treat eosinophil-associated hypersensitivity diseases such as bronchial asthma by locally administering a topical anesthetic (Gleich et al., U.S. Pat. No. 5,510,339 issued Apr. 23, 1996.) Gleich et al. proposes that topical anesthetics such as N-arylamide, aminoalkylbenzoate, lidocaine, lidocaine hydrocholoride, prilocaine, etidocaine and their pharmaceutically acceptable salts are suitable for this purpose. Gleich et al. suggest that additional topical anesthetics treat disease by inhibiting the activity of eosinophil-active cytokines where the disease is intranasal inflammation, nasal polyps, paranasal sinus inflammation, allergic rhinitis, and other diseases. Such additional proposed topical anesthetics claimed by Gleich et al include procaine, chloroprocaine, dyclonine, tetrecaine, benoxinate, proparacaine, meprylcaine, and piperocaine. (Gleich et al., U.S. Pat. No. 5,631,267 issued May 20, 1997).

Gleich also proposed methods to treat eosinophil-assoicated pathology, such as bronchial asthma, by co-administering a topical anesthetic and a glucocorticoid. Still further topical anesthetics suggested are bupivacaine and dibucaine. Suggested glucocorticoids include beclomethasone, cortisol, cortisone, dexamethasone, flumethosone, fluocinolone, fluticasone, meprednisone, methylprednisolone, prednisolone, triamcinolone, amcinonide, desonide, desoximetasone, or pharmaceutical salts thereof. (Gleich et al., U.S. Pat. No. 5,837,713 issued Nov. 17, 1998).

Gleich et al. also propose methods to treat neutrophil-associated pulmonary diseases such as COPD, chronic bronchitis (CB), cystic fibrosis, α-1 anti-trypsin deficiency, pulmonary emphysema, adults respiratory distress syndrome (ARDS) or idiopathic pulmonary fibrosis by locally administering a topical anesthetic (Gleich et al., U.S. Publication No. 20030171402). Direct application of local anesthetics to airways has been explored to treat asthma, cough and bronchoconstriction induced by intubation. While these agents appear to be effective in preventing reflex bronchoconstriction, they can also induce bronchoconstriction. This paradoxical effect limits the utility of these agents in treating cough and local airway inflammation, especially in asthmatic patients. Adrenergic β-agonists (such as epinephrine and albuterol) have been used in conjunction with local anesthetics. While adrenergic β-agonists will cause bronchodilation, data reported in the literature does not address the effect of this class of drugs on bronchoconstriction that occurs shortly (within minutes) of treatment with local anesthetics. Further, recent studies indicate that β-agonists, a mainstay of asthma therapy, appear to lose their effectiveness in some patients when used on a regular basis. The result is that instead of acting to relieve bronchoconstriction, these drugs increase bronchial hyperresponsiveness. In the past, researchers have suggested that desensitization to β-agonists is responsible for this untoward effect. Thus, alternatives to β-agonists are needed.

While in the relative long term, anesthetics work well at anesthetizing the pulmonary system, in the first critical moments after administration, the anesthetics actually causes bronchorestriction actually increasing discomfort and sometimes causing panic in the patient who perceives that the discomfort is actually worsening.

Accordingly, there is a need for a treatment which will treat a patient suffering from a pulmonary condition while avoiding untoward effects of local anesthetics, that is, without exacerbating the condition in the first moments after administration of the local anesthetic.

There is also a need for individualized therapy which can be tailored to the unique physiological response of a patient thereby optimizing the outcome for that patient.

### SUMMARY OF THE INVENTION

The present invention includes compositions and methods for pulmonary conditions, especially pulmonary conditions characterized by persistent cough, using combinations of muscarinic receptor antagonists and local anesthetics. A persistent cough can accompany acute, subacute as well as chronic conditions, including degenerative conditions. The compositions and methods of the invention are useful in the treatment of many pulmonary conditions, including but not limited to, acute respiratory distress syndrome (ARDS), α-1 antitrypsin deficiency, asbestosis/dust disease, asthma, bronchiectasis, bronchopulmonary dysplasia (BPD), bronchoconstriction induced by intubation, cancer of the lungs, chronic bronchitis, chronic cough, chronic obstructive pulmonary disease (COPD), common cold, cystic fibrosis, emphysema, Farmer's lung (also known as extrinsic allergic alveolitis, hypersensitivity pneumonitis and other immunologically mediated inflammatory disease of the lung involving the terminal airways related to the inhalation of biological dusts), hantavirus, histoplasmosis, influenza, legionellosis, lung cancer, lymphangioleiomyomatosis, lung transplantation, organ donation, pertussis, pleurisy, pneumonia, pneumothorax, primary alveolar hypoventilation syndrome, pulmonary alveolar proteinosis, pulmonary embolus, pulmonary fibrosis, pulmonary hypertension, respiratory distress syndrome, respiratory syncytial virus, sarcoidosis, severe acute respiratory syndrome (SARS), smoker's cough, spontaneous pneumothorax, or tuberculosis. The compositions and methods of the invention can be used either alone or in conjunction with other therapies for the pulmonary conditions.

In one embodiment, dry particles are prepared having the combinations in the same particles. In other embodiment, blends of dry particles may be employed and administered pulmonarily simultaneously in the same breath. In still another embodiment, blends of dry particles may be employed and administered in sequence. In yet another embodiment, liquid formulations comprising the combination of muscarinic receptor antagonists and local anesthetics are employed. In such embodiments, the liquid formulations are administered pulmonarily, for example, by nebulization, spray or other means know to those skilled in the art. The combinations are administered simultaneously or in sequence. If administered in sequence, the muscarinic receptor antagonist is administered before the local anesthetic. In certain embodiments where the muscarinic receptor antagonist is administered before the local anesthetic, the muscarinic receptor antagonist is administered about a few seconds to about 15 minutes before the administration of the local anesthetic. The administration may be repeated as the condition indicates. The administration may be alternated with other treatments.

### BRIEF DESCRIPTION OF THE DRAWINGS

FIG. 1 is a graph indicating PenH levels over Time after Dosing (in minutes) following administration of saline or lidocaine (1.25 or 2.5 mg) in guinea pigs immunized against ovalbumin.

FIG. 2 is a graph indicating PenH levels over Time after Dosing (in minutes) which demonstrate the ability of the muscarinic receptor antagonist IpBr to block the lidocaine induced increase in PenH.

FIG. 3 is a bar graph showing PenH levels as indicator of the ability of a) saline, b) lidocaine, c) IpBr and d) lidocaine & IpBr to potentiate methacholine-induced bronchoconstriction.

FIG. 4 is a bar graph showing PenH levels as an indicator or the ability of a) lidocaine alone, b) lidocaine & IpBr and c) lidocaine & epinephrine to block methacholine-induced bronchoconstriction within 30 minutes of treatment.

FIG. 5 is a bar graph showing PenH levels as an indicator of bronchoconstriction after administering a) saline, b) lidocaine, c) lidocaine & epinephrine, and d) lidocaine & IpBr in about 10 to about 15 minutes after the termination of anesthesia in guinea pigs.

### DETAILED DESCRIPTION OF THE INVENTION

The present invention relates to compositions and methods for pulmonary conditions, especially pulmonary conditions characterized by persistent cough, using combinations of muscarinic receptor antagonists and local anesthetics. A persistent cough can accompany acute, subacute as well as chronic conditions, including degenerative conditions. The compositions and methods of the invention are useful in the treatment of many pulmonary conditions, including but not limited to, acute respiratory distress syndrome (ARDS), α-1 antitrypsin deficiency, asbestosis/dust disease, asthma, bronchiectasis, bronchopulmonary dysplasia (BPD), bronchoconstriction induced by intubation, cancer of the lungs, chronic bronchitis, chronic cough, chronic obstructive pulmonary disease (COPD), common cold, cystic fibrosis, emphysema, Farmer's lung (also known as extrinsic allergic alveolitis, hypersensitivity pneumonitis and other immunologically mediated inflammatory disease of the lung involving the terminal airways related to the inhalation of biological dusts), hantavirus, histoplasmosis, influenza, legionellosis, lung cancer, lymphangioleiomyomatosis, lung transplantation, organ donation, pertussis, pleurisy, pneumonia, pneumothorax, primary alveolar hypoventilation syndrome, pulmonary alveolar proteinosis, pulmonary embolus, pulmonary fibrosis, pulmonary hypertension, respiratory distress syndrome, respiratory syncytial virus, sarcoidosis, severe acute respiratory syndrome (SARS), smoker's cough, spontaneous pneumothorax, or tuberculosis. The compositions and methods of the invention can be used either alone or in conjunction with other therapies for the pulmonary conditions. For example, treatment of certain pulmonary conditions require alternating between inducing productive coughing at certain times and suppressing coughing at other times. For example, compositions and methods of the invention can be used alternatively in conjunction with chest percussion, back clapping and body positioning to drain lung secretions in pulmonary conditions such as, but not limited to, cystic fibrosis and COPD.

In one embodiment, dry particles are prepared having the combinations in the same particles. In other embodiment, blends of dry particles may be employed and simultaneously pulmonarily administered in the same breath. In still another embodiment, blends of dry particles may be employed and administered in sequence. In yet another embodiment, liquid formulations comprising the combination of muscarinic receptor antagonists and local anesthetics are employed. In such embodiments, the liquid formulations are administered pulmonarily, for example, by nebulization, spray or other means know to those skilled in the art. The combinations are administered simultaneously or in sequence. If administered in sequence, the muscarinic receptor antagonist is administered before the local anesthetic. In certain embodiments where the muscarinic receptor antagonist is administered before the local anesthetic, the muscarinic receptor antagonist is administered about 5 to about 15 minutes before the administration of the local anesthetic. The administration may be repeated as the condition indicates. Examples of suitable muscarinic receptor antagonist include but are not limited to ipratropium bromide, trospium chloride and tiotropium. Suitable local anesthetics include but are not limited N-arylamide, aminoalkylbenzoate, benoxinate, bupivacaine, chloroprocaine, dibucaine, dyclonine, etidocaine, lidocaine, lidocaine hydrocholoride, proparacaine, mepivacaine, meprylcaine, piperocaine, prilocaine, procaine, tetrecaine and their pharmaceutically acceptable salts. Many other suitable local anesthetics are available. Table 1 lists exemplary local anesthetics and dosing information but is not intended to be limiting.

TABLE 1

| | Relative Onset | |
|---|---|---|
| | of action with | |
| Agent | infiltration | Maximum one-time dose |
| Lidocaine | Fast | 4.5 mg/kg |
| | | (30 mL in average [70-kg] adult) |
| Mepivacaine | Fast | 7 mg/kg |
| (Carbocaine, | | (30 mL in average [70-kg] adult) |
| Polocaine) | | |
| Bupivacaine | Moderate | 2 mg/kg |
| (Marcaine, | | (50 mL in average [70-kg] adult) |
| Sensorcaine) | | |

Muscarinic receptor antagonists (MRA) are a class of compounds that have been shown to cause bronchodilation under defined conditions. This drug class is used clinically to treat COPD and asthma. However, these drugs are not used, or recognized as rescue medications for the rapid relief of bronchoconstriction. While not wishing to be limited to a single theory, Applicants believe that local anesthetics (LA; such as lidocaine (also known as lignocaine), bupivacaine, mepivacaine, and procaine) cause bronchoconstriction by increasing acetylcholine (ACH) levels near bronchial smooth muscle, resulting in bronchoconstriction via activation of muscarinic receptors. It has been discovered that concomitant administration of muscarinic receptor antagonist with lidocaine rapidly prevents bronchoconstriction by directly blocking muscarinic receptors. Animal models are used to demonstrate that administration of muscarinic receptor antagonists (MRA) either before or simultaneously with a local anesthetic prevents bronchoconstriction which is induced by local anesthetics. For example, animal models were used to demonstrate that co-administration of muscarinic receptor antagonists (MRA) prevented bronchoconstriction induced by local anesthetics. The combination therefore permits the use of local anesthetics in treatment of any of the pulmonary conditions listed above, in particular, chronic persistent cough, asthma and COPD. As a test, Applicants studied the effects of lidocaine in guinea pigs. In one study discussed in more detail in the Exemplification section, a guinea pig model of human asthma was employed to test ipratropium bromide (IpBr) as a prototype MRA and lidocaine as the prototype local anesthetic. As mentioned above, lidocaine can cause bronchoconstriction in asthmatic humans. The guinea pigs were sensitized, anesthetized and administering a liquid to the airways which contributed to a transient increase in PenH lasting about 2 minutes. Administration of lidocaine increased the duration of the elevated PenH and the magnitude of the lidocaine-induced PenH increase was dose-dependent. Yet, concomitant administration of IpBr blocked the effect of lidocaine on PenH. (See Example 1 for details and description of Figures).

Of course, for the optimal treatment of pulmonary conditions, the drugs of choice must reach the appropriate location in the lung. Accordingly, the drug or drugs are delivered pulmonarily through nebulization, metered dose inhalers, dry powder inhaler and the like.

In one embodiment the drugs, for example a muscarinic receptor antagonists (MRA) and a local anesthetic, are formulated in dry particles. Applicant's assignee has filed numerous patent applications drawn to various innovations in the spray drying art as it relates to improvements in the production of dry particles. See for example, U.S. Publication No. 20030180283 published Sep. 25, 2003 entitled “Method and Apparatus for Producing Dry Particles,” which is related to PCT application with the same title PCT/U.S.03/08398 (published as WO03/080028), entitled “Method and Apparatus for Producing Dry Particles,” U.S. Publication No. 20030017113 published Jan. 23, 2003 entitled “Control of process humidity to produce porous particles,” and U.S. Publication No. 2003222364 with the same title published Dec. 4, 2003. For example, in the above mentioned U.S. Publication No. 20030017113, Applicant found that particles can be formed which possess targeted aerodynamic properties by controlling the moisture content of a drying gas and contacting the liquid droplets which are formed with the drying gas, thereby drying the liquid droplets to form spray dried particles. The entire teachings of all referenced patent applications, patent publications, journals and any other references throughout this entire application are incorporated herein by reference. When employing dry particle technology where the local anesthetic and the MRA are combined in the same particle (LA-MRA combination) simultaneous and efficient delivery of local anesthetic-MRA combinations to the lungs is achieved. The LA-MRA combination is well-suited to the production of combination drug formulations due to the fact that LA-MRA combination particles are comprised of drug(s) and excipients in a single formulation. In a preferred embodiment, LA-MRA combination particles produced via a simple one-step unit operation process (spray-drying) contain the same ratio of drug(s) and excipients within each particle. In addition to manufacturing advantages, this ensures that drug(s) embedded within LA-MRA combination particles are simultaneously delivered to the same micro-environmental sites in the lungs, enabling their synergistic effects. LA-MRA combination particles possesses advantages such as ease of powder dispersion and efficiency of delivery, enabling the use of simple, breath-actuated inhalers that can deliver in excess of 70 percent of a nominal dose to the lungs over a wide range of inhalation flow rates and volumes in a single inhalation. Finally, LA-MRA combination particles can be readily formulated possessing a wide range of chemical properties, such as hydrophilicities and hydrophobicities, utilizing a variety of excipients that are approved and/or safe for inhalation, such as sugars, amino acids, surfactants, and the like. In one embodiment, the particles are relatively uniform in size as measured by fine particle fraction.

In other embodiments, the local anesthetic, MRA and/or LA-MRA combination are combined with inert carriers. Suitable inert carriers include simple carbohydrates or polysaccharides. For example, local anesthetic, MRA and/or LA-MRA combinations may be combined lactose blends, that are comprised of distinct micronized particles blended with coarse lactose particles to aid in dispersion. In such embodiments, particle blends are engineered to have the desired heterogeneity or relatively homogeneity. In so engineering the particles, the device used to administer is taken into account to optimize the performance of the particles. Other combinations would be obvious to one skilled in the art.

In further embodiments, the local anesthetic, MRA and/or LA-MRA combination are combined with inert carriers in a form other than a particle, either dry or micronized.

### EXEMPLIFICATION

### Example 1

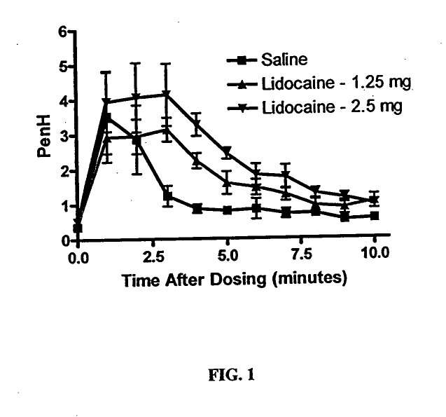

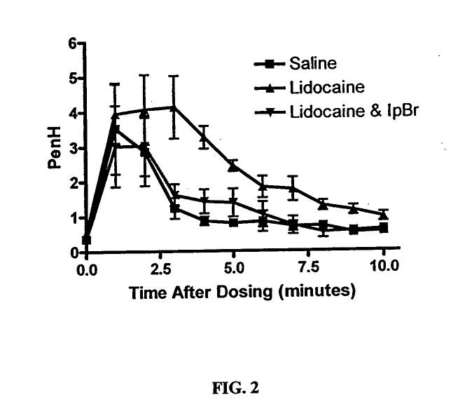

Applicants have studied the effects of lidocaine in ova-sensitized guinea pigs. Sensitizing guinea pigs to ovalbumin leads to increased numbers of eosinophils in airway tissues and has been used as a model system of human asthma. For this study, ipratropium bromide (IpBr) was used as a prototype MRA and lidocaine was used as a prototype local anesthetic. This study explored bronchoconstriction caused by lidocaine. Guinea pigs were immunized against ovalbumin. To evaluate the ability of lidocaine to induced bronchoconstriction in this model, guinea pigs were a) anesthetized only (control), b) anesthetized and instilled with 200 μL of saline, or c) anesthetized and instilled with 1.25 or 2.5 mg of lidocaine in 200 μL of saline. Immediately after dosing animals were placed in BUXCO whole body plethysmograph chambers and pulmonary function monitored. The processes of anesthetizing animals and administering a liquid to the airways each contribute to a transient increase in PenH lasting about 2 minutes (FIG. 1; data not shown for the control treatment). Administration of lidocaine increased the duration of the elevated PenH to approximately 6 minutes. Furthermore the magnitude of the lidocaine-induced PenH increase was dose-dependent (see FIG. 1 which shows increased PenH following administration of saline or lidocaine (1.25 or 2.5 mg) in guinea pigs immunized against ovalbumin). Concomitant administration of IpBr blocked the effect of lidocaine on PenH with the PenH values being similar to those observed in animals receiving only saline (see FIG. 2 which shows the ability of the muscarinic receptor antagonist IpBr to block the lidocaine induced increase in PenH).

### Example 2

Applicants conducted additional studies. In order to administer liquids or dry powders directly to the airways of small rodents and guinea pigs they must be lightly anesthetized.

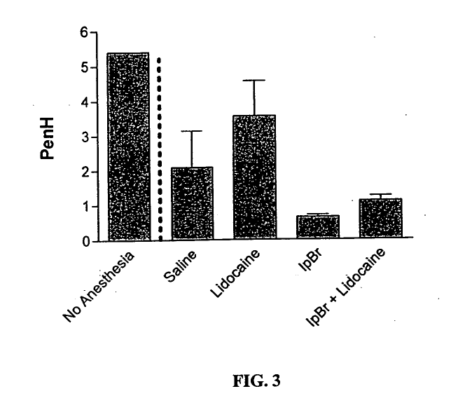

While inhaled anesthetics can cause a brief transient increase in PenH, Applicants observed that isoflurane paradoxically reduces the bronchoconstrictive effects of methacholine for a short time—approximately 20 to 30 minutes (see FIG. 3). During the course of these investigations, applicants also discovered a short time interval following termination of isoflurane anesthesia (15 and 30 minutes) where lidocaine enhanced the bronchoconstrictive action of methacholine in guinea pigs. This model was utilized to evaluate the ability of a MRA to block effects of lidocaine and rapidly inhibit the effects of the muscarinic agonist methacholine.

Lidocaine versus Lidocaine & IpBr

Guinea pigs were anesthetized with isoflurane and one of the following treatments instilled into the airway using a Penn Century device designed for liquid instillation: a) saline; b) lidocaine; c) IpBr or d) lidocaine and IpBr. The volume of liquid instilled was between 200-300 μL for each treatment group. Between 20 and 30 minutes following treatment all animals received methacholine by nebulization. Treatment with lidocaine caused an increase PenH relative to guinea pigs receiving only saline. IpBr was effective in blocking methacholine induced bronchoconstriction, including in guinea pigs receiving lidocaine (see FIG. 3 which shows that IpBr attenuates lidocaine's ability to potentiate methacholine-induced bronchoconstriction).

Lidocaine Versus Lidocaine & IpBr Versus Lidocaine & Epinephrine

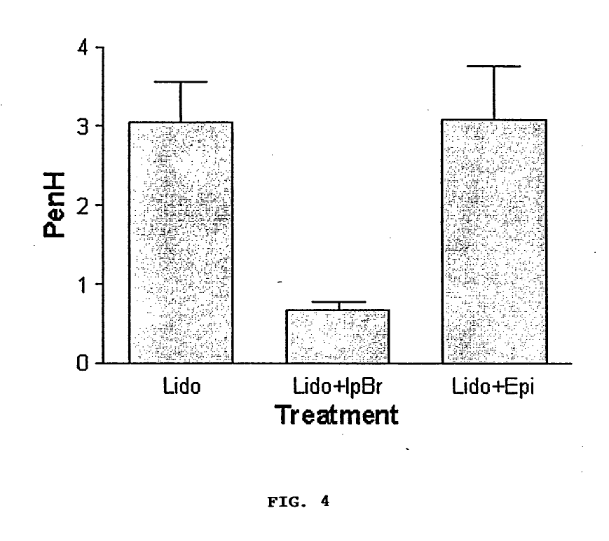

The relative ability of IpBr to block methacholine and lidocaine induced bronchoconstriction was compared to epinephrine, an adrenergic agonist known to cause bronchodilation. For this study, animals were anesthetized and one of the following instilled into the airway: a) lidocaine, b) lidocaine and epinephrine, or c) lidocaine and IpBr. Guinea pigs were then exposed to methacholine within 20-30 minutes of terminating anesthesia. Results from this study are shown in FIG. 4 which indicates that IpBr, but not epinephrine, blocks methacholine-induced bronchoconstriction out to 30 minutes of treatment. There are no differences in PenH among Lidocaine, Lidocaine & epinephrine and saline (not shown).

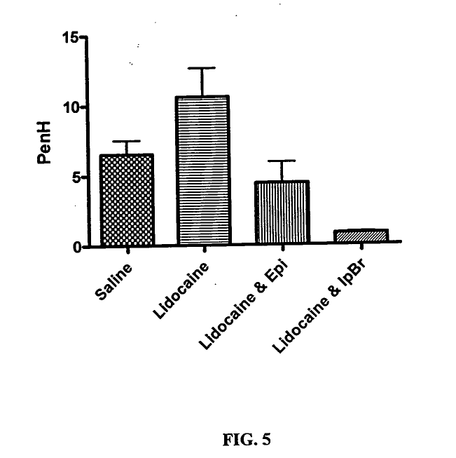

A second study was conducted to evaluate the ability of IpBr and epinephrine to reduce lidocaine/methacholine induced bronchoconstriction earlier relative to the termination of isoflurane. Saline, lidocaine and lidocaine plus epinephrine all result in an increase in PenH over baseline. The increase in PenH caused by lidocaine alone relative to saline is not typically observed after 15 minutes, as seen in the previous experiment. Thus, IpBr can greatly reduce PenH back to baseline levels even with lidocaine administration. Guinea pigs were anesthetized with isoflurane and treated with: a) saline; b) lidocaine; c) lidocaine plus epinephrine; or d) lidocaine plus IpBr. Approximately 10-15 minutes later each guinea pig was exposed to methacholine by nebulization. Results are shown in FIG. 5 which show the comparison of lidocaine, lidocaine & epinephrine, and lidocaine & IpBr on bronchoconstriction shortly after the termination of anesthesia in guinea pigs. While epinephrine reduced PenH to levels similar to those observed in saline treated animals, IpBr was much more effective reducing PenH by approximately 90% relative to animals receiving only lidocaine.

### Example 3

Initial formulation efforts for local anesthetics (for example, lidocaine, bupivacaine, mepivacaine) and muscarinic receptor antagonists (for example, trospium and IpBr) are summarized in the tables below. Based on these results and previous efforts producing powders, combination formulations containing up to about 90% local anesthetic and up to about 8% MRA are suitable.

TABLE 2
Non-limiting examples of Lidocaine formulations

| Load | | | VMGD (μm) | | FPF_{TD} (%) | |
|---|---|---|---|---|---|---|
| (%) | Composition | | 1 bar | 2 bar | <5.6 μm | <3.4 μm |
| 50 | DPPC/Leu/ | 5/37.2/57.8 | 7.2 | 5.3 | 45 | 14 |
| | LidoHCl | | | | | |
| 25 | DPPC/Leu/ | 10/61.1/28.9 | 7.2 | 5.3 | 50 | 18 |
| | LidoHCl | | | | | |
| 50 | DPPC/Leu/ | 10/32.2/57.8 | 9.1 | 6.9 | 39 | 15 |
| | LidoHCl | | | | | |
| 50 | Leu/LidoHCl | 42.2/57.8 | 5.6 | 4.6 | 62 | 32 |
| 40 | Leu/LidoHCl | 53.8/46.2 | 7.5 | 5.9 | 35 | 18 |
| 25 | Leu/LidoHCl | 71.1/28.9 | 9.3 | 7.7 | 48 | 23 |
| 90 | Leu/Lido | 10/90 | 22.4 | 15.7 | 19 | 9 |
| 50 | Leu/Lido | 50/50 | 7.5 | 5.6 | 30 | 14 |

TABLE 3
Non-limiting examples of bupivacaine formulations

| | | |
|---|---|---|
| Load | VMGD (μm) | FPF_{TD} (%) |

| (%) | Composition | | 1 bar | 2 bar | <5.6 μm | <3.4 μm |
|---|---|---|---|---|---|---|
| 25 | Leu/BupiHCl | 71.8/28.2 | 6.9 | 6.2 | 56 | 33 |
| 25 | DPPC/Leu/ | 10/61.8/28.2 | 6.9 | 6.3 | 65 | 27 |
| | BupiHCl | | | | | |

TABLE 4
Non-limiting examples of mepivacaine formulations

| Load | | | VMGD (μm) | | FPF_{TD} (%) | |
|---|---|---|---|---|---|---|
| (%) | Composition | | 1 bar | 2 bar | <5.6 μm | <3.4 μm |
| 50 | Leu/MepiHCl | 42.6/57.4 | 5.8 | 5.3 | 48 | 22 |
| 50 | DPPC/Leu/ | 10/32.6/57.4 | 9.1 | 6.9 | 59 | 26 |
| | MepiHCl | | | | | |
| 50 | DPPC/Leu/ | 5/37.6/57.4 | 7.0 | 5.4 | 55 | 20 |
| | MepiHCl | | | | | |

TABLE 5
Non-limiting examples of trospium formulations

| | | | | | FPF_{TD} | |
|---|---|---|---|---|---|---|
| Load | | | VMGD (μm) | | (%) (ACI-2) | |
| (%) | Composition | | 1 bar | 2 bar | <5.6 μm | <3.4 μm |
| 5 | Leu/DPPC/trospium | 85/10/5 | 8.6 | 6.5 | 79 | 59 |
| 5 | Leu/DPPC/DSPC/ | 85/5/5/5 | 8.2 | 6.6 | 80 | 56 |
| | trospium | | | | | |
| 5 | Leu/DPPC/trospium | 90/5/5 | 7.1 | 6.0 | 75 | 53 |

E1 = 70:20:10 DPPC:sodium citrate:calcium chloride
E2 = 60:20:20 DPPC:DPPE:lactose
E3 = 35:35:30 DPPC:DSPC:leucine

Other suitable formulations which could be adapted for use in the invention can be found in U.S. Ser. No. 10/392,333 the entire teachings of which are incorporated herein by reference.

| | | RODOS | | FPF (std. dev.) | |
|---|---|---|---|---|---|
| | Formulation | 1 bar | 2 bar | <5.6 μm | <3.4 μm |
| 1 | (E1, 1% IpBr) | 10.53 | 8.89 | 0.61 (0.03) | 0.25 (0.01) |
| 2 | (E1, 4% IpBr) | 10.89 | 9.74 | 0.59 (0.03) | 0.25 (0.01) |
| 3 | (E1, 8% IpBr) | 10.00 | 8.48 | 0.48 (0.01) | 0.17 (0.00) |
| 4 | (E2, 1% IpBr) | 9.06 | 7.67 | 0.58 (0.00) | 0.26 (0.01) |
| 5 | (E2, 4% IpBr) | 8.84 | 7.56 | 0.62 (0.00) | 0.29 (0.02) |
| 6 | (E2, 8% IpBr) | 9.27 | 8.14 | 0.42 (0.01) | 0.17 (0.00) |
| 7 | (E3, 1% IpBr) | 7.38 | 7.12 | 0.37 (0.05) | 0.12 (0.02) |
| 8 | (E3, 4% IpBr) | 7.03 | 6.40 | 0.50 (0.03) | 0.18 (0.02) |
| 9 | (E3, 8% IpBr) | 7.16 | 5.99 | 0.43 (0.01) | 0.15 (0.01) |
| 10 | (E3, 1% IpBr) | 13.97 | 9.88 | 0.44 (0.10) | 0.19 (0.04) |
| 11 | (E3, 4% IpBr) | 12.19 | 8.58 | 0.39 (0.11) | 0.15 (0.04) |
| 12 | (E3, 8% IpBr) | 16.51 | 10.50 | 0.38 (0.01) | 0.13 (0.00) |

What is claimed is:

1. A composition for treating pulmonary conditions comprising at least one muscarinic receptor antagonist and at least one local anesthetic.

2. The composition of claim 1, wherein the pulmonary conditions are characterized by persistent cough.

3. The composition of claim 1, wherein the pulmonary condition is selected from the group consisting of an acute condition, a subacute condition and a chronic condition.

4. The composition of claim 1, wherein the pulmonary condition is a degenerative condition.

5. The composition of claim 1, wherein the pulmonary condition is selected from the group consisting of acute respiratory distress syndrome (ARDS), α-1 antitrypsin deficiency, asbestosis/dust disease, asthma, bronchiectasis, bronchopulmonary dysplasia (BPD), bronchoconstriction induced by intubation, cancer of the lungs, chronic bronchitis, chronic cough, chronic obstructive pulmonary disease (COPD), common cold, cystic fibrosis, emphysema, Farmer's lung (also known as extrinsic allergic alveolitis, hypersensitivity pneumonitis and other immunologically mediated inflammatory disease of the lung involving the terminal airways related to the inhalation of biological dusts), hantavirus, histoplasmosis, influenza, legionellosis, lung cancer, lymphangioleiomyomatosis, lung transplantation, organ donation, pertussis, pleurisy, pneumonia, pneumothorax, primary alveolar hypoventilation syndrome, pulmonary alveolar proteinosis, pulmonary embolus, pulmonary fibrosis, pulmonary hypertension, respiratory distress syndrome, respiratory syncytial virus, sarcoidosis, severe acute respiratory syndrome (SARS), smoker's cough, spontaneous pneumothorax, or tuberculosis.

6. The composition of claim 1, wherein said at least one muscarinic receptor antagonist is selected from the group consisting of ipratropium bromide, trospium chloride and tiotropium.

7. The composition of claim 1, wherein said at least one local anesthetic is selected from the group consisting N-arylamide, aminoalkylbenzoate, benoxinate, bupivacaine, chloroprocaine, dibucaine, dyclonine, etidocaine, lidocaine, lidocaine hydrocholoride, proparacaine, mepivacaine, meprylcaine, piperocaine, prilocaine, procaine, tetrecaine and their pharmaceutically acceptable salts.

8. The composition of claim 1 further comprising additional therapies for the pulmonary conditions.

9. The composition of claim 1, wherein said composition is suitable for pulmonary administration.

10. The composition of claim 1, wherein said at least one muscarinic receptor antagonist and said at least one local anesthetic are administered simultaneously via pulmonary administration.

11. The composition of claim 1, wherein said at least one muscarinic receptor antagonist and said at least one local anesthetic are administered in the same breath.

12. The composition of claim 1, wherein said at least one muscarinic receptor antagonist and said at least one local anesthetic are present in a powder.

13. The composition of claim 12, wherein said powder is selected from the group consisting of dry particles, micronized particles, or combinations thereof.

14. The composition of claim 12, wherein said at least one muscarinic receptor antagonist and said at least one local anesthetic are present in the same dry particles.

15. The composition of claim 12, wherein said at least one muscarinic receptor antagonist and said at least one local anesthetic are present in the same micronized particles.

16. The composition of claim 12, wherein said at least one muscarinic receptor antagonist and said at least one local anesthetic are in the separate dry particles.

17. The composition of claim 12, wherein said at least one muscarinic receptor antagonist and said at least one local anesthetic are in the separate micronized particles.

18. The composition of claim 16, wherein said separate dry particles are blended.

19. The composition of claim 17, wherein said separate micronized particles are blended.

20. The composition of claim 1, wherein said at least one muscarinic receptor antagonist and said at least one local anesthetic prepared as a liquid formulation.

21. The composition of claim 20, wherein the liquid formulation is pulmonarily administered.

22. The composition of claim 21, wherein the pulmonary administration is selected from the group consisting of nebulization and spray.

23. The composition of claim 21, wherein the administration is repeated.

24. A method for treating pulmonary conditions comprising administering at least one muscarinic receptor antagonist and at least one local anesthetic.
